(19) United States
(12) Patent Application Publication
(10) Pub. No.: US 2004/0102367 A1
(43) Pub. Date: May 27, 2004

(54) Gene expression system based on chimeric receptors

(76) Inventors: Fred H Gage, La Jolla, CA (US); Steven T Suhr, La Jolla, CA (US)

Correspondence Address: FOLEY & LARDNER, P.O. BOX 80278, SAN DIEGO, CA, 92138-0278, US

(21) Appl. No.: 10/181,325

(22) PCT Filed: Feb. 23, 2001
(86) PCT No.: PCT/US01/05750

(51) Int. Cl. A61K 38/17

(52) U.S. Cl. 514/12

(57) ABSTRACT

The invention provides a system for modulating the expression of a target gene in a subject wherein a defined response element for a DNA binding domain modulates expression of said target gene. The invention system comprises two chimeric proteins, each containing the dimerization domain of a member of the steroid/thyroid hormone nuclear receptor superfamily, one of which is non-endogenous to the subject. In addition, the first chimeric protein contains a DNA binding domain to which the target gene is responsive and the second chimeric protein contains a transcription modulating domain, such as a transcription activator or a transcription repressor. In one embodiment of the invention, two invention systems form a dimer having the properties of a native heterodimer or homodimer. In another embodiment, only the first chimeric protein contains a DNA binding domain and only the second chimeric protein contains a transcription activating domain. The functional entity formed by association of the first and second chimeric proteins can be designed to transactivate transcription by complexing with a DNA binding recognition site that does not have the 2-half site format common to response elements for members of the steroid/thyroid hormone nuclear receptor superfamily. Thus, certain of the invention systems cannot functionally interact with endogenous proteins in the way that wild type receptors do. The invention further provides nucleic acid sequences encoding the invention chimeric proteins, cells containing such nucleic acid sequences, and methods for using the invention chimeric proteins to modulate expression of one or more non-endogenous genes in a subject organism.

### FIELD OF THE INVENTION

The present invention relates to methods in the field of recombinant DNA technology, and products related thereto. In a particular aspect, the invention relates to methods for modulating the expression of genes in mammalian or non-mammalian systems, and products useful therefor.

### BACKGROUND OF THE INVENTION

The ability to control the expression of genes introduced into cells and organisms is important in many areas of biology and medicine. For instance, methods that allow the intentional manipulation of gene expression would facilitate the analysis of genes whose production cannot be tolerated constitutively or at certain stages of development. They would also be valuable for clinical applications such as gene therapy protocols, where the expression of a therapeutic gene must be regulated in accordance with the needs of the patient.

To be of broad benefit such systems will preferably be indifferent to endogenous factors and will preferably be activated by non-endogenous nontoxic drugs. The components of the system preferably will not interfere with endogenous cellular pathways. In many cases inducibility is a desirable feature, but requires that the system have a minimal basal activity in the inactive state, while rapidly achieving high levels of expression of the target gene upon activation. Additional desirable features of such a system are rapid return to the inactive state, e.g., by clearance of an inducing molecule from the system of the subject, and a response that is proportional to the concentration of the inducing molecule so that quantitative as well as qualitative problems can be addressed.

Steroid/thyroid hormone nuclear receptors control gene expression and have been used in the field of genetic engineering as a tool for manipulating and controlling development and other physiological processes. For example, applications for regulated gene expression in mammalian systems include inducible gene targeting, overexpression of toxic and teratogenic genes, anti-sense RNA expression, and gene therapy (see, for example, R. Jaenisch, Science 240:1468-1474, 1988). For cultured cells, glucocorticoids and other steroids have been used to induce the expression of a desired gene.

As another means for controlling gene expression in mammalian systems, an inducible tetracycline regulated system has been devised and utilized in transgenic mice, whereby gene activity is induced in the absence of tetracycline and repressed in its presence (see, e.g, Gossen et al. PNAS 89:5547-5551,1992; Gossen et al., TIBS 18:471-475, 1993; Furth et al., PNAS 91:9302-9306, 1994; and Shockett et al., PNAS 92:6522-6526, 1995). However, disadvantages of the inducible tetracycline system include the requirement for continuous administration of tetracycline to repress expression and the slow clearance of antibiotic from bone, a side-effect that interferes with regulation of gene expression. While this system has been improved by the recent identification of a mutant tetracycline repressor that acts conversely as an inducible activator, the pharmacokinetics of tetracycline may hinder its use during development when a precise and efficient “on-off” switch is essential (see, e.g., Gossen et al., Science 268:1766-1769, 1995).

Certain insect steroid/thyroid hormone nuclear receptors have also been studied. The Drosophila melanogaster ecdysone receptor (EcR) (M. R. Koelle et al., Cell 67:59-77, 1995) is unlike the estrogen, androgen, and other homodimeric vertebrate steroid hormone nuclear receptors because it requires a heterologous dimer partner for functional transactivation. The obligate dimer partner, the product of the ultraspiracle (Usp) gene (V. C. Henrich et al., Nuc. Acids Res. 18: 4143-4148, 1990; T. P. Yao et al., supra, 1992; T. P. Yao et al., Nature 366:476-479, 1993), is an insect homolog of the mammalian retinoid X receptor (RXR) proteins found in vertebrates and other mammalian species. RXRs have been characterized as regulatory dimer partners of many mammalian class II steroid/thyroid hormone nuclear receptors, such as the thyroid hormone receptors, the retinoic acid receptors, and the vitamin D receptor (reviewed in Mangelsdorf and Evans, Cell 83:841-850, 1995; D. J. Mangelsdorf et al., Cell 83: 835-839, 1995). RXR is also capable of functioning as a dimer partner of EcR.

Usp and RXR share a significant degree of sequence homology and some functional similarities; however, in formation of heterodimers with EcR, RXR interacts differently than Usp. One primary difference is that formation of EcR+RXR heterodimers is more highly stimulated by the ecdysteroid ligand muristerone A (murA) than by 20-hydroxyecdysone (20-Ec), while formation of EcR+Usp heterodimers is potently stimulated by 20-hydroxyecdysone (K. S. Christopherson et al., Proc Natl Acad Sci USA 89:6314-6318, 1982; H. E. Thomas et al., Nature 362:471-475, 1993). A second difference is in the way that ligand promotes efficient formation of EcR+Usp and EcR+RXR heterodimer complexes and concomitant binding to ecdysone response elements (EcREs). MurA stimulates EcR+Usp binding of EcREs approximately 3 to 7-fold over levels without ligand, but EcR+RXR complexes are completely dependent on ligand for heterodimerization. As yet another difference between RXR and Usp, EcR+RXR complexes bind to EcREs at only 10-40% the level of EcR+Usp complexes (Christopherson et al., supra 1982; Thomas et al., supra 1993; Yao et al., supra, 1992 & 1993). This suggests that the affinity of EcR for its natural dimer partner, Usp, is significantly greater than its affinity for RXR. A number of additional ecdysone receptors are known in the art as being responsive to an applied non-endogenous chemical inducer enabling external control of expression of the gene controlled by the receptor (See, for example, PCT/GB96/01195 and PCT/US98/17723).

Another approach to regulate gene expression is derived from studies on the mechanism of action of immunosuppressive agents and relies on chemical induction of protein dimerization. Chemical inducers of dimerization have been used to transactivate transcription of target genes. However, such chemical inducers of dimerization have the potential to affect the immune system and require structure-guided modification to avoid unwanted immunosuppression in subjects (E. Saez et al., Curr. Opin. Biotechnol. 8(5):608-16, 1997).

Accordingly, there is a need in the art for new and improved systems to precisely modulate the expression of non-endogenous genes in mammalian systems. For example, there is a need in the art for new and better methods of using steroid/thyroid hormone nuclear receptors that require a dimer partner for functional transactivation of transgene expression for use in somatic gene therapy and for laboratory models thereof.

### BRIEF DESCRIPTION OF THE INVENTION

In accordance with the present invention, there are provided chimeric receptor systems for modulating the expression of a target gene in a subject. Invention systems are based on the discovery that a functional chimeric transcription factor for ligand-dependent modulation of gene expression can be constituted in vivo by protein-protein interaction of separate chimeric proteins that each contain the dimerization domain of a dimerizing protein, specifically a member of the steroid/thyroid hormone nuclear receptor superfamily, and further comprise one of two components necessary to form a transcription factor, i.e., a transcription modulation domain and a DNA binding domain. Association of the dimerization domains in the two chimeric proteins, and, hence, “formation” of the transcription factor by juxtaposition of its two component parts, optionally in the presence of a non-endogenous ligand for the system, will allow the system to operate in subjects without interference from endogenous receptors and ligands.

Accordingly, in accordance with one embodiment of the present invention, there are provided systems for modulating the expression of a target gene associated with a defined response element in a subject. Invention systems comprise a first chimeric protein comprising at least one dimerization domain of a first member of the steroid/thyroid hormone nuclear receptor superfamily and at least one DNA binding domain, and a second chimeric protein comprising at least one dimerization domain of a second member of the steroid/thyroid hormone nuclear receptor superfamily and at least one transcription modulating domain, wherein the first and second chimeric proteins associate to form a functional entity under substantially physiological conditions, and wherein response of the response element to said DNA binding domain modulates expression of the target gene.

In accordance with another embodiment of the present invention, there are provided one or more isolated nucleic acids encoding invention systems, as well as vectors and cells containing such nucleic acid(s). In one embodiment, the isolated nucleic acid jointly encodes the first and second chimeric proteins, for example with an internal ribosomal entry site located between the nucleotides encoding the first and second chimeric proteins.

In accordance with yet another embodiment of the present invention, there are provided systems for modulating the expression of a target gene associated with a defined response element in a subject, wherein the system comprises a first chimeric protein comprising at least one dimerization domain of a first member of the steroid/thyroid hormone nuclear receptor superfamily and at least one DNA binding domain, and a second chimeric protein comprising at least one dimerization domain of a second member of the steroid/thyroid hormone nuclear receptor superfamily and at least one transcription modulating domain, wherein at least one of the receptors is non-endogenous to said subject and the first and second chimeric proteins associate to form a functional entity under substantially physiological conditions in the presence or absence of a non-endogenous ligand. Response of the response element to the DNA binding domain in the functional entity modulates expression of the target gene in the subject.

In accordance with still another embodiment of the present invention, there are provided method(s) for modulating the expression of one or more target genes in a subject that contains one or more inducible DNA constructs encoding an invention system and DNA encoding one or more target gene products under the control of a response element to which the DNA binding domain in the invention system is responsive. In this embodiment, invention method(s) comprises subjecting the subject to conditions suitable to induce expression of the system, thereby modulating expression of the target gene(s).

In accordance with a further embodiment of the present invention, there are provided method(s) for modulating the expression of one or more target genes in a subject containing an invention system and DNA encoding one or more target gene products under the control of a response element to which the DNA binding domain in the invention system is responsive. In this embodiment, invention method(s) comprises administering to the subject an effective amount of ligand for the system, thereby modulating expression of the target gene(s).

In accordance with a further embodiment of the present invention, there are provided system(s) for modulating the expression of a target gene associated with a defined response element in a subject. Invention system(s) comprises a first chimeric protein consisting of a DNA binding domain and a dimerization domain of a first member of the steroid/thyroid hormone nuclear receptor superfamily, and a second chimeric protein consisting of a transcription modulating domain and a dimerization domain of a second member of the steroid/thyroid hormone nuclear receptor superfamily, wherein one of the receptors is non-mammalian and the first and second chimeric proteins associate to form a functional entity under substantially physiological conditions in the presence of a non-mammalian ligand. Response of the response element associated with the target gene to DNA binding domain in invention system(s) modulates expression of the target gene(s).

### BRIEF DESCRIPTION OF THE FIGURES

FIG. 1 is a schematic of the chimeric proteins which comprise the invention modulation system.

FIG. 2 is a schematic of how the components of the invention modulation system interact in the absence and presence of ligand, wherein the black bars represent a response element, the open bars represent a first chimeric protein (comprising a dimerization domain and a DNA binding domain), the darkly shaded bars represent a second chimeric protein (comprising a dimerization domain and a transcription modulating domain), and the small circles represent molecules of ligand. As shown on the left hand side of the figure, in the absence of ligand, the first and second chimera do not associate, whereas (as shown on the right hand side of the figure) in the presence of ligand, the first and second chimera associate, thereby promoting transcription of the gene(s) associated with the response element.

FIG. 3 presents a schematic of VHB, GR and GU constructs. BE numbers refers to amino acids from the Bombyx ecdysone receptor hinge domain (“h”) and hormone binding domain (HBD) used in construction; R numbers refer to amino acids from the human RXR alpha protein used in construction of GR; and U numbers refer to amino acids from the Drosophila Usp protein used in construction of GU. VP16 sequences are a 77 amino acid region of the HSV VP16 protein transactivation domain; GAL4 sequences are a 95 amino acid domain of the GAL4 protein capable of tightly and specifically binding its cognate operator DNA response element of 17 base-pairs.

FIG. 4 presents data demonstrating the operability of tandem chimeric proteins as described herein using different amounts of plasmids encoding VHB (VP 16 activation domain operatively associated with the Bombyx ecdysone receptor ligand binding domain) and GR (GAL4 DNA binding domain operatively associated with the RXR ligand bonding domain) in a transient transfection assay using 1 μM tebufenozide as ligand, and measuring luciferase activity 48 hrs. after transfection and stimulation. The reporter plasmid is two tandem Gal4 operators coupled with a minimal Tk promoter and driving the firefly luciferase gene. Units are Relative Units.

FIG. 5 presents data illustrating the properties of RXR-USP chimeric proteins. Black indicates RXR protein regions, gray indicates Drosophila USP protein regions. R or U numbers indicate the amino acids corresponding to human RXR alpha or Drosophila Usp used in the construction of chimeras. By combining different regions of both proteins and empirically assaying, chimeric variants with novel ligand responsive properties were found. Units are Relative Units. Numbers at the end of each bar indicate fold induction (calculated by dividing the induced level by the unstimulated level for each ligand). GRU variant 1.2 demonstrates good response to all ligands (both 20-OH ecdysone and tebufenozide) with a relatively low baseline, however, the baseline is not down to the level of GR or GR-like chimeras with super-low uninduced expression levels. To further reduce basal transactivation while retaining a strong response to 20-OH ecdysone, point mutations within the GRU1.2 hormone binding domain were made and tested (see FIG. 6).

FIG. 6 presents data generated by a point mutant of GRU1.2, referred to herein as GRU1.2D-3, wherein the cysteine at position 329 (or dUsp sequence) has been converted to glycine. This mutant displays a significantly lower basal level of transactivation than its parent chimer, yet it retains a high level response to both 20-OH ecdysone and tebufenozide. The relative increase multiple transient transfection experiments routinely fall within 50-100-fold induction.

FIG. 7 schematically illustrates exchange of the Gal4 DNA-binding domain of GRU1.2D-3 with the 260 amino acid bacterial tetracycline repressor protein DNA binding region results in a new constuct called VIRTU that allows regulation by both ecdysteroids and tetracycline analogs. This construct functions essentially equivalently to GRU variants with respect to induction by ecdysteroids and ecdysteroid agonists such as tebufenozide.

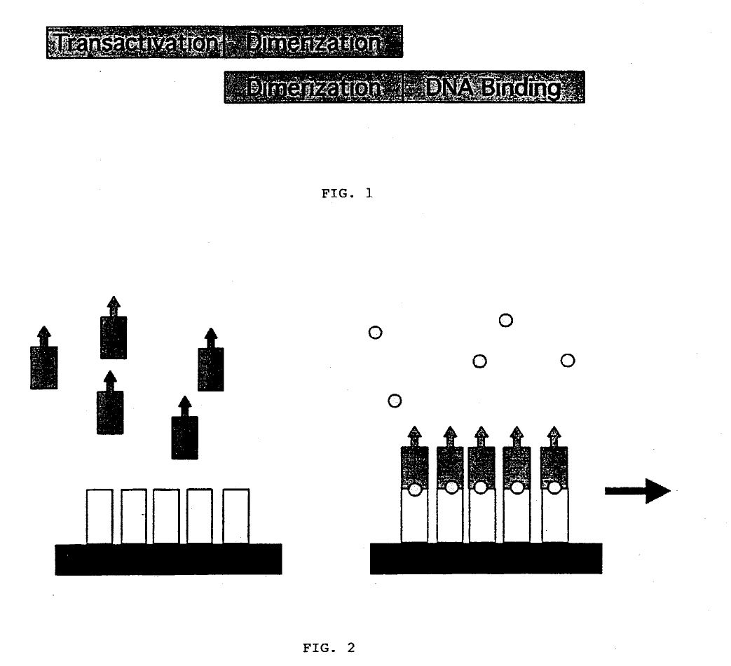

### DETAILED DESCRIPTION OF THE INVENTION

In accordance with the present invention, there are provided systems for modulating the expression of a target gene associated with a defined response element in a subject. The invention system comprises a first chimeric protein comprising at least one dimerization domain of a first member of the steroid/thyroid hormone nuclear receptor superfamily and at least one DNA binding domain, and a second chimeric protein comprising at least one dimerization domain of a second member of the steroid/thyroid hormone nuclear receptor superfamily and at least one transcription modulating domain. The first and second chimeric proteins associate to form a functional entity under substantially physiological conditions, wherein response of the response element to the DNA binding domain modulates expression of the target gene. Association of a functional entity can take place either in the presence or absence of a ligand for the system. Preferably the ligand, if present, is non-endogenous to the subject.

Optionally, two invention systems may associate to form a dimer, for example either a heterodimer or a homodimer. In certain cases, invention systems may also form dimers with native members of the steroid/thyroid hormone nuclear receptor superfamily.

In a presently preferred embodiment of the invention system, the first chimeric protein consists essentially of a DNA binding domain and a first dimerization domain that are non-endogenous to the subject to which it is administered and the second chimeric protein consists essentially of a transactivation modulating domain and a second dimerization domain. For example, in a mammalian subject, the DNA binding domain and first dimerization domain can be selected to be non-mammalian, for example, derived from a plant or insect. Alternatively, for administration to a plant, at least one of the DNA binding domain and the first dimerization domain can be mammalian. It is also contemplated, as one of numerous possibilities, that a non-mammalian DNA binding domain can be combined with a mammalian dimerization domain in the invention systems.

The DNA binding domain and transactivation domains can be located anywhere within the respective chimeric proteins. Preferably, the DNA binding domain is located at the carboxyl terminus of the first chimeric protein and the transactivation modulating domain is located at the amino terminus of the second chimeric protein. Optionally, upon association of the two chimeric proteins, the transactivation modulating domain and the DNA binding domain are brought into spatial juxtaposition with one another to “form” a chimeric transcription factor functional for ligand-dependent modulation of gene expression.

At least one dimerization domain in the invention system is optionally selected to be ligand responsive, so that interaction between the dimerization domains is preferably dependent on the presence or absence of ligand for the system. In mammalian subjects, modulation of expression of a target gene can be achieved by inducing expression of one chimeric protein (if the other is constitutively produced), of both chimera, by introducing a non-endogenous (e.g., non-mammalian) ligand to the subject, or by removing such ligand from the subject. In any event, expression of target genes is preferably controlled by the addition to the system of ligand therefor or removal of ligand from the system.

Invention chimeric proteins associate to form functional entities under a variety of conditions, including those at or near physiological conditions in the subject into which they are introduced, with the particular physiological conditions depending upon whether the subject is a plant, a mammal, an amphibian, etc. (e.g., in saline at body temperature in mammals). Those of skill in the art will understand that association of proteins can be influenced by manipulation of a variety of physical parameters, such as are disclosed in McPherson, Eur. J. Biochem., 189:1-23, 1990, which is incorporated herein by reference in its entirety.

As used herein with reference to an invention system containing a first and second chimeric protein, the term “functional entity” means that the associated first and second chimeric proteins (either as monomers or as dimers) possess at least some of the biological function of wild type receptors which promote transactivation of a promoter operatively associated with a suitable response element, i.e., one responsive to the invention system. For example, two invention systems can dimerize, or an invention system can dimerize with a native member of the steroid/thyroid hormone nuclear receptor superfamily to modulate transactivation of gene(s) whose expression is controlled by a response element that responds to the two DNA binding domains in the dimer (i.e., a 2-site response element), optionally in the presence of ligand. Alternatively, the functional entity formed by association of the first and second chimeric proteins in the invention system can, as a monomer (i.e., without association with another invention system or with a member of the steroid/thyroid hormone nuclear receptor superfamily) modulate transactivation of gene(s) whose expression is controlled by a response element that is responsive to the single DNA binding domain contained therein. Even in the situation wherein the response element is a 2-site response element, such as a hormone response element, the invention system can, as a monomer, transactivate at least a low level of expression. For example, an invention system wherein at least one chimeric protein comprises a Bombyx mori DNA binding domain can modulate the expression of a gene under the control of a Bombyx ecdysone response element. However, for transactivation of nucleic acid operatively associated with a 2-site response element, it is preferred that the invention systems function as a homodimer, or a heterodimer.

Therefore, the term “functional entity,” as applied to the invention system, means that the two chimeric proteins possess a cooperative function. For example, in a functional entity the two dimerization domains interact with each other in a manner appropriate to substantially provide one or more of the functions in the functional entity that are present in a wild type member of the steroid/thyroid hormone nuclear receptor superfamily.

As used herein, the phrase “member(s) of the steroid/thyroid hormone nuclear receptor superfamily” (also known as “intracellular receptors” or “the nuclear receptor superfamily”) refers to hormone binding proteins that operate as ligand-dependent transcription factors, including identified members of the steroid/thyroid hormone nuclear receptor superfamily for which specific ligands have not yet been identified (referred to in the art as “orphan receptors”). Members of the steroid/thyroid hormone nuclear receptor superfamily are characterized by the presence of five domains: N-terminal or activation domain (A/B), DNA binding domain (C), hinge domain (D), ligand binding domain (E), and C-terminal domain (F) (Evans, R. Science 240:889-895, 1988).

Each chimeric protein in the invention system is required to contain a dimerization domain of a member of the steroid/thyroid hormone nuclear receptor superfamily. As used herein, “dimerization domain” means a region derived from one or more members of the steroid/thyroid hormone nuclear receptor superfamily containing a sequence of amino acids that functions to cause dimerization of two invention chimeric proteins. In members of the steroid/thyroid hormone nuclear receptor superfamily, the dimerization domain is generally located within the region of the receptor molecule that is commonly referred to as including the D, E and F domains, or is referred to as the “D-E-F” domain. Typically the dimerization domain, therefore, includes the complete ligand binding domain (E), or a portion thereof, and may optionally include all or part of the hinge domain (D) and/or the C-terminal region (F) of a member of the steroid/thyroid nuclear receptor superfamily, or a functional equivalent thereof. In some cases the dimerization domain may include at least a portion of the DNA binding domain itself.

The ligand binding domain employed for the preparation of invention chimeric proteins can be either endogenous or non-endogenous to a subject to whom the invention system is to be administered, with the former including ligand binding domains that are modified to be non-responsive to ligands endogenous or native to the subject. In embodiments wherein the ligand binding domain is derived from non-mammalian member(s) of the steroid/thyroid hormone nuclear receptor superfamily, the ligand binding domains are preferably derived from the carboxy-terminal portion of non-mammalian members. Exemplary members that are not normally present in mammalian cells include insect, avian, amphibian, reptilian, fish, plant, bacteria, viral and fungal (including yeast) members of the steroid/thyroid hormone nuclear receptor superfamily, and the like. The ligand binding domain can also be derived from other mammalian systems, such as DXR or SXR.

Exemplary members of the steroid/thyroid hormone receptor superfamily (including the various isoforms thereof) whose dimerization domains are useful in the practice of the present invention include steroid receptors such as glucocorticoid receptor (GR), mineralocorticoid receptor (MR), estrogen receptor (ER), progesterone receptor (PR), androgen receptor (AR), vitamin D₃ receptor (VDR), and the like; plus retinoid receptors, such as the various isoforms of retinoic acid receptor (e.g., RARα, RARβ or RARγ, the various isoforms of retinoid X (or 9-cis retinoic acid) receptor (e.g., RXRα, RXRβ, or RXRγ, various isoforms of peroxisome proliferator-activated receptors (e.g., PPARα, PPARγ, PPARδ) and the like; thyroid hormone receptor (T₃R), such as TRα, TRβ, and the like; steroid and xenobiotic receptor (SXR, see for example, Blumberg et al., Genes Dev (1998) 12(20):3195-205), RXR-interacting proteins (RIPs; see, e.g., Seol et al., Mol Endocrinol (1995) 9(1):72-85; Zavacki et al., Proc Natl Acad Sci USA (1997) 94(15):7909-14) including farnesoid X receptor (FXR; see for example, Forman et al., Cell (1995) 81(5:687-93; Hanley et al., J Clin Invest (1997) 100(3):705-12, O'Brien et al., Carcinogenesis (1996) 17(2):185-90), pregnenolone X receptor (PXR; see for example, Schuetz et al., Mol Pharmacol (1998) 54(6):1113-7), liver X receptor (LXR, see, e.g., Peet et al., Curr Opin Genet Dev (1998) 8(5):571-5), BXR (Blumberg et al., Genes Dev (1998) 12(9):1269-77), insect derived receptors such as the ecdysone receptor (EcR), the ultraspiracle receptor (see, for example, Oro et al., in Nature 347:298-301 (1990)), and the like; as well as other gene products which, by their structure and properties, are considered to be members of the superfamily, as defined hereinabove, including the various isoforms thereof (see, e.g., Laudet, V., J Mol Endocrinol (1997) 19(3:207-26).

Examples of orphan receptors contemplated for use herein include HNF4 (see, for example, Sladek et al., Genes & Development 4:2353-2365 (1990)), the COUP family of receptors (see, for example, Miyajima et al., in Nucleic Acids Research 16:11057-11074 (1988), and Wang et al., Nature 340:163-166 (1989)), COUP-like receptors and COUP homologs, such as those described by Mlodzik et al., Cell 60:211-224 (1990) and Ladias et al., Science 251:561-565 (1991), orphan receptor (OR1; see, e.g., Feltkamp et al., J Biol Chem (1999) 274(15):10421-9), the insect derived knirps and knirps-related receptors, short heterodimer partner (SHP; see, e.g., Seol et al., Mol Cell Biol (1997) 17(12):7126-31), hepatocyte nuclear receptor 4 (HNF4), constitutive androstane receptor (CAR; see, e.g., Forman et al., Nature (1998) 395(6702):612-5), and the like.

In a presently preferred embodiment of the present invention, one of the dimerization domains is derived from an ecdysone receptor (EcR), for example, a Drosophila EcR (DEcR) or a Bombyx EcR (BEcR). It has been discovered that the dimerization preferences of wild type receptors are substantially retained in the chimeric proteins of invention systems, such that association of two invention chimera to form a functional entity follows the dimerization preferences of native members of the steroid/thyroid hormone nuclear receptor superfamily. For example, when one of the chimeric proteins contains the dimerization domain from an ecdysone receptor, it is presently preferred that the other chimeric protein contain a dimerization domain from a retinoid X receptor or an Ultraspiracle receptor.

Additional, exemplary ecdysone receptors for use in invention systems include those derived from dipteran species such as Drosophila melanogaster (M. R. Koelle, 1995), and the like, and those derived from lepidopteran species such as Bombyx mori (Swevers et al., Insect Biochem. Molec. Biol., 25(7):857-866, 1995), Choristoneura fumiferana (Palli et al., Insect Biochem. Molec. Biol., 26(5):485-499, 1996), Manduca sexta (Fujiwara et al., Insect Biochem. Molec. Biol., 25(7):845-856, 1995), Aedes aegypti (Cho et al., Insect Biochem Molec. Biol., 25:19-27, 1995), Chorinomus tentans (Imhof et al., Insect Biochem. Molec. Biol., 25:115-124, 1993), Heliothis virescens (PCT/GB96/01195), Spodoptera exigua, and the like.

In a presently preferred embodiment according to the present invention, at least one of the chimeric proteins comprises a dimerization affinity enhancing domain in addition to the dimerization domain. The presently preferred affinity enhancing domain is the hinge region (D) i.e., the fragment bounded by the ligand binding domain (E) and DNA binding domain (C) of the native Bombyx mori receptor (BEcR), specifically, about 27 amino acid residues (i.e. amino acid residues 283-309 of BECR), which are sufficient to confer high affinity for complex formation with an endogenous dimer partner (see U.S. patent application Ser. No. 08/891,298, filed Jul. 10, 1997, copending herewith, which is incorporated herein by reference in its entirety).

The first chimeric protein in the invention system contains a DNA binding-domain. DNA-binding domains contemplated for use in the preparation of invention chimeric proteins are well known in the art and are typically obtained from DNA-binding proteins (e.g., transcription factors). The term “DNA-binding domain” is understood in the art to refer to an amino acid sequence that is able to bind to DNA (A. Klug, Ann N Y Acad Sci, 758:143-160, 1995). As used herein, the term “DNA-binding domain” encompasses a minimal peptide sequence of a DNA-binding protein up to the entire length of a DNA-binding protein, so long as the DNA-binding domain functions to associate with a particular response element, as a monomer, homomultimer, or a heteromultimer, preferably a monomer or homodimer. The DNA-binding domain can be positioned at either the carboxyl or amino terminus of the first chimeric protein in the invention system.

DNA-binding domains are known to function heterologously in combination with other functional domains while maintaining the ability to bind the natural DNA recognition sequence (see, e.g., Brent and Ptashne, Cell, 43:729-736, 1985). For example, with respect to steroid/thyroid hormone nuclear receptors, DNA-binding domains are interchangeable, thereby providing numerous chimeric receptor proteins (see, e.g., U.S. Pat. No. 4,981,784; and R. Evans, Science, 240:889-895, 1988). “DNA-binding protein(s)” contemplated for use herein belong to the well-known class of proteins that are able to directly bind DNA and facilitate initiation or repression of transcription. Exemplary DNA-binding proteins contemplated for use herein include transcription control proteins (e.g., transcription factors and the like; see, for example, Conaway and Conaway, Transcription Mechanisms and Regulation, Raven Press Series on Molecular and Cellular Biology, Vol. 3, Raven Press, Ltd., New York, N.Y., 1994; T. Boulikas, Critical Reviews in Eukaryotic Gene Expression, 4(2&3):117-321, 1994; A. Klug, Gene 135:83-92, 1993; W. M. Krajewska, Int. J. Biochem., 24:1885-1898,1992.)

Transcription factors contemplated for use herein as a source of such DNA binding domains include, e.g., homeobox proteins, zinc finger proteins, hormone receptors, helix-turn-helix proteins, helix-loop-helix proteins, basic-Zip proteins (bZip), β-ribbon factors, and the like. See, for example, S. Harrison, “A Structural Taxonomy of DNA-binding Domains,” Nature, 353:715-719. Homeobox DNA-binding proteins suitable for use herein include, for example, HOX, STF-1 (Leonard et al., Mol. Endo., 7:1275-1283, 1993), Antp, Mat α-2, INV, and the like. See, also, Scott et al. Biochem. Biophys. Acta, 989:25-48, 1989. It has been found that a fragment of 76 amino acids (corresponding to amino acids 140-215 described in Leonard et al., 1993) containing the STF-1 homeodomain binds DNA as tightly as wild-type STF-1. Suitable zinc finger DNA-binding proteins for use herein include Zif268, GLI, XFin, and the like. See also, Klug and Rhodes, Trends Biochem. Sci., 12:464, 1987; Jacobs and Michaels, New Biol., 2:583, 1990; and Jacobs, EMBO J., 11:4507-4517, 1992.

The DNA-binding domain(s) used in the invention chimeric proteins can be obtained from a member of the steroid/thyroid hormone nuclear receptor superfamily, or are substantially the same as those obtained from a member of the superfamily. The DNA-binding domains of substantially all members of the steroid/thyroid hormone nuclear receptor superfamily are related. Such domains consist of 66-68 amino acid residues, and possess about 20 invariant amino acid residues, including nine cysteines. Members of the superfamily are characterized as proteins which contain these 20 invariant amino acid residues. The highly conserved amino acids of the DNA-binding domain of members of the superfamily are as follows:

| | |
|---|---|
| Cys - X - X - Cys - X - X - Asp* - X - Ala* - X - Gly* - | (SEQ ID NO:1) |
| X - Tyr* - X - X - X - X - Cys - X - X - Cys - Lys* - X - | |
| Phe - Phe - X - Arg* - X - X - X - X - X - (X - X - )Cys - | |
| X - X - X - X - X - (X - X - X - )Cys - X - X - X - Lys - X - | |
| X - Arg - X - X - Cys - X - X - Cys - Arg* - X - X - | |
| Lys* - Cys - X - X - X - Gly* - Met; | |

wherein X designates non-conserved amino acids within the DNA-binding domain; an asterisk denotes the amino acid residues which are almost universally conserved, but for which variations have been found in some identified hormone receptors; and the residues enclosed in parenthesis are optional residues (thus, the DNA-binding domain is a minimum of 66 amino acids in length, but can contain several additional residues).

Preferably, only the first chimeric protein and not the second chimeric protein in the invention system contains a DNA binding domain. In this embodiment of the present invention, the second chimeric protein (which lacks a DNA binding domain) cannot interact productively with a member of the steroid/thyroid hormone nuclear receptor superfamily (e.g., an endogenous one) to complete the 2-half site requirement for DNA binding. Consequently, in this scenario, the invention system produces a functional entity that complexes with only one half of a 2-half site DNA recognition site.

Alternatively, when interaction with 2-half site response elements is to be avoided, a preferred DNA binding domain is the GAL4 DNA binding domain, which does not interact with a 2-half site DNA recognition site. The DNA binding domain of the yeast GAL4 protein comprises at least the first 74 amino terminal amino acids thereof (see, for example, Keegan et al., Science 231:699-704, 1986). Preferably, the first 90 or more amino terminal amino acids of the GAL4 protein will be used, for example, the 147 amino terminal amino acid residues of yeast GAL4. The GAL4 DNA binding domain is particularly effective when combined in the first chimeric protein with the ligand binding domain of a retinoid X receptor or an Ultraspiracle receptor because a dimer partner containing this combination cannot functionally interact with endogenous proteins as can the native retinoid X receptor.

Another DNA binding domain preferred for use in the practice of the present invention is the Tet operon. The tetracycline inducible system is well-known in the art (see, e.g, Gossen et al. (1992) Proc. Natl. Acad. Sci. 89, 5547-5551; Gossen et al. (1993) TIBS 18, 471-475; Furth et al. (1994) Proc. Natl. Acad. Sci. 91, 9302-9306; and Shockett et al. (1995) Proc. Natl. Acad. Sci. 92, 6522-6526). Use of the Tet operon as DNA binding domain in the first chimeric protein is particularly effective when combined with the ligand binding domain of a Bombyx ecdysone receptor.

The second chimeric protein in the invention system comprises a transcription modulating domain in addition to a dimerization domain. Preferably only the second chimeric protein and not the first chimeric protein comprises a transcription modulating domain. Transcription modulating domains are of two types, those that activate transcription of a gene sequence operatively associated with a response element that is responsive to the invention system (i.e., transcription activation domains) and those that repress or de-activate transcription of a gene sequence operatively associated with a response element that is responsive to the invention system (i.e., transcription repression domains). The transcription modulating domain can be located anywhere within the second chimeric protein, but is preferably located at the amino terminus thereof.

The ability of the invention system to activate transcription of such a target gene is generally enhanced when the transcription modulating domain in the second chimeric protein is a transcription activation domain. Transcription activation domains contemplated for use in the practice of the present invention are well known in the art and can readily be identified by those of skill in the art. Such transcription activation domains are typically derived from transcription factors and comprise a contiguous sequence that functions to activate gene expression when associated with a suitable DNA-binding domain and, optionally, a suitable ligand binding domain.

Suitable activation domains can be obtained from a variety of sources, e.g., from the N-terminal region of members of the steroid/thyroid hormone nuclear receptor superfamily, from transcription factor activation domains, such as, for example, VP16, GAL4, NF-kB or BP64 activation domains, and the like (See, for example, M. Manteuffel-Cymborowska, Acta Biochim Pol. 46(1):77-89, 1999; T. Tagami et al., Biochem Biophys Res. Commun. 253(2):358-63,1998, W. Westin, Adv Pharmacol, 47:89-112, 2000). The activation domain presently preferred for use in the practice of the present invention is obtained from the C-terminal region of the VP16 protein, and is known as VP16τ. For activating or enhancing transcription of a target gene, it is presently preferred that the second chimeric protein comprises the VP16τ activation domain and both the hinge domain and the ligand binding domain of a Bombyx ecdysone receptor as the second dimerization domain.

The ability of the invention system to repress transcription of a target gene is generally enhanced when the transcription modulating domain in the second chimeric protein is a transcription repressor domain. Transcription repressor domains specifically include those that repress transactivation of gene expression by nuclear receptors by affecting the interaction of the ligand and/or functional entity with a response element that is otherwise responsive to the functional entity, either as a monomer or as a dimer, as well as those that “de-activate” by inhibiting association of the first and second chimeric proteins. Exemplary transcription repressor domains suitable for use as the transcription modulating domain in the second chimeric protein include RAFT, CREM, MECP-2, SMRT, NcoR, mSin3A, RAR, TR, SMRTR, and the like.

In accordance with another embodiment of the present invention, there are provided one or more isolated nucleic acid(s) encoding invention chimeric protein(s) and cells containing such one or more isolated nucleic acids. Cells containing invention isolated nucleic acid(s) can be either mammalian or non-mammalian, for example, plant, bacterial, viral and fungal (including yeast) cells, and the like.

In accordance with yet another embodiment of the present invention, there are provided methods for modulating the expression of one or more target gene(s) in a subject containing:

1) an invention system as described herein that forms a functional entity upon association with a ligand, and

2) DNA encoding one or more target gene products under the control of a response element to which the DNA binding domain is responsive.

In this embodiment, invention methods comprise administering to the subject an effective amount of ligand for the invention system. The ligand for use in the practice of the present invention is preferably non-endogenous to the subject treated for modulation of expression of one or more target genes, i.e., for enhancing or repressing expression of the respective gene product(s). The target gene is generally non-endogenous, but may also be endogenous if either increased or decreased production of an endogenous gene product is desired.

In a presently preferred embodiment of the present invention, one of the dimerization domains in the functional entity formed by association of the two chimeric proteins is from an insect species, and the preferred ligand for use in the invention method is an insect hormone, non-mammalian analog, or mimic thereof. Preferably, the dimerization domain in one invention chimeric protein is derived from a diptera species (e.g., the Drosophila ecdysone receptor) or a lepidoptera species (e.g., the Bombyx ecdysone receptor), and the dimerization domain contained in the other invention chimeric protein is derived from either the ultraspiracle protein or a retinoid X receptor (or a chimeric combination of the two). Invention systems containing either one of these combinations will complex with an ecdysone response element in the presence of such a ligand for the insect receptor.

In accordance with still another embodiment of the present invention, there are provided methods for modulating the expression of one or more target gene(s) in a subject containing:

1) DNA encoding an invention system under the control of an inducible promoter, and

2) DNA encoding one or more target gene products under the control of a response element to which the DNA binding domain is responsive.

In this embodiment of the invention, the invention method comprises administering to the subject an effective amount of an inducer for the invention system.

As employed herein, the terms “modulate” and “modulating” refer to the ability of a given functional entity or a dimer of such functional entities to activate/deactivate and/or up-regulate/down-regulate transcription of target nucleic acid(s) (e.g., non-endogenous nucleic acid(s)), relative to transcription of target nucleic acid in the absence of the functional entity or dimer of such functional entities.

The actual effect of an invention functional entity or dimer thereof on the transcription of non-endogenous or endogenous nucleic acids will vary depending on the particular combination of transcription modulating domain, dimerization domain(s) and/or specific members of the steroid/thyroid hormone nuclear receptor superfamily in the two chimeric proteins, on the presence or absence of specific ligand for the ligand binding domain(s) employed in the chimeric proteins, on the regulatory element (e.g., response element) with which the DNA binding domain and/or the functional entity interacts, on the presence or absence of additional components of a receptor complex (e.g., co-activators, co-repressors), and the like. It is specifically contemplated within the scope of the present invention that modulation includes repression of expression of one or more genes.

In accordance with yet another embodiment of the present invention, there are provided methods for modulating the expression of one or more target genes (i.e., either activating or repressing) in a cell line. In this embodiment the invention method, the cell line contains:

1) an invention system and

2) a DNA construct comprising the target gene under the control of a response element with which the invention system interacts, wherein the response element controls expression of the target gene.

In such a cell line, the invention method comprises administering to the cell line an effective amount of a non-endogenous ligand for the invention system to either enhance or repress expression of the target gene. This embodiment of the invention can be practiced either in vitro or in vivo.

In accordance with a further embodiment of the present invention, there are provided methods for modulating the expression of one or more genes in a subject containing an endogenous response element controlling expression of one or more genes. The invention method in this situation comprises introducing to the subject an invention system containing the first and second chimeric proteins, wherein the functional entity formed by the chimeric proteins interacts with the endogenous response element, thereby modulating expression of the gene(s) dependent on the presence of ligand therefor. Generally, the ligand will not be endogenous to the subject, thereby providing control over expression of the gene product(s). If the invention system is encoded by an inducible DNA construct, the modulating can further comprise inducing expression of the nucleic acid(s) encoding the components of the invention system.

In accordance with another embodiment of the present invention, there are provided methods for modulating the expression of one or more genes in a subject containing an endogenous ligand and an endogenous response element controlling expression of one or more genes. In this situation, the invention method comprises introducing to the subject an invention system that interacts with the endogenous ligand and with the endogenous response element, thereby modulating expression of the one or more gene(s). If the invention system is encoded by an inducible DNA construct, the modulation will be controlled by subjecting the subject to conditions suitable to cause expression of the first and second chimeric proteins. For example, the subject can be administered an effective amount of an inducer for the DNA construct. This embodiment of the invention is especially useful for controlling expression of a non-endogenous gene that is under the control of an endogenous response element when the ligand for the invention functional entity is also endogenous.

Response elements contemplated for use in the practice of the present invention (relating to modulation of the expression of non-endogenous genes in a subject) include native, as well as modified response elements. For example, since invention functional entities can function as either homodimers or as heterodimers, any response element that is responsive to an invention functional entity, in the form of a homodimer or heterodimer, is contemplated for use in the invention methods described herein. As is readily recognized by those of skill in the art, invention functional entities (whether in the form of a homodimer or a heterodimer) as well as invention monomers can bind to a response element having an inverted repeat motif (i.e., two or more half sites in mirror image orientation with respect to one another), to a response element having a direct repeat motif, to palindromic response elements, and the like.

Response elements contemplated for use in the practice of the present invention include elements responsive to the invention functional entity and/or to the DNA binding domain contained therein. In a preferred embodiment of the present invention, such elements are non-endogenous response elements not normally present in the cells of the host. One class of non-endogenous response elements contemplated for use herein includes hormone response elements that modulate transcription of non-endogenous nucleic acid when associated with the DNA binding domain of an invention receptor peptide. Such response elements are referred to herein as being “defined” or “responsive to” the DNA binding domain in one or more invention systems.

Response elements useful in conjunction with invention functional entities are well known in the art. As readily recognized by those of skill in the art, the response element employed will vary as a function of the protein units incorporated into the invention system(s). Thus, for example, retinoic acid receptor response elements are composed of at least one direct repeat of two or more defined half sites separated by a spacer of five nucleotides. The spacer nucleotides can independently be selected from any one of A, C, G or T. Each half site of the 2-site response elements contemplated for use in the practice of the invention comprises the sequence:

-RGBNNM- (SEQ ID NO:2),

wherein

R is selected from A or G;

B is selected from G, C, or T;

each N is independently selected from A, T, C, or G; and

M is selected from A or C;

with the proviso that at least 4 nucleotides of said -RGBNNM- (SEQ ID NO:3) sequence are identical with the nucleotides at corresponding positions of the sequence -AGGTCA- (SEQ ID NO:4). Such response elements employed in the practice of the present invention can optionally be preceded by Nₓ, wherein x falls in the range of 0 up to 5.

For example, thyroid hormone receptor response elements can be composed of the same half site repeats, with a spacer of four nucleotides. Alternatively, palindromic constructs as have been described in the art are also functional as thyroid receptor response elements.

When the invention system contains a GAL4 DNA binding domain, the response element selected for use in the invention system is a GAL4 response element. Exemplary GAL4 response elements are those containing the palindromic 17-mer:

5′-CGGAGGACTGTCCTCCG-3′ (SEQ ID NO:5),

such as, for example, 17MX, as described by Webster et al., in Cell 52:169-178 (1988), as well as derivatives thereof. Additional examples of suitable response elements include those described by Hollenberg and Evans in Cell 55:899-906 (1988); or Webster et al. in Cell 54:199-207 (1988).

Ecdysone response element sequences are preferred for use herein with invention systems containing an ecdysone receptor DNA binding domain. Such response elements function in a position- and orientation-independent fashion. The native ecdysone response element has been previously described, see, e.g., Yao et al., Cell, 71:63-72, 1992.

It is a particular advantage of the present invention that any DNA binding recognition function can be substituted in place of those having the 2-half-site requirement of members of the steroid/thyroid hormone nuclear receptor superfamily. For this reason, the invention system and methods can be used to transactivate endogenous target genes in a ligand specific manner.

Certain nucleic acid constructs contemplated for use in one aspect of the present invention include promoters and/or regulatory elements operatively associated with non-endogenous nucleic acids. In one embodiment of the present invention, the invention system, in the presence of a ligand therefor, complexes with the response element and activates transcription of one or more non-endogenous nucleic acids, including non-endogenous reporter gene(s). For example, an invention system containing a DNA binding domain and/or dimerization domains derived from RXR and EcR will transactivate an ecdysone response element-containing promoter in the presence of the hormone ecdysone, or the synthetic analog, muristerone A.

In the invention methods, the response element is operatively associated with one or more non-endogenous target gene(s) whose expression it is desirable to control. As used herein, the phrase “operatively associated with” refers to the functional relationship of DNA with regulatory and effector sequences of nucleotides, such as promoters, enhancers, transcriptional and translational stop sites, and other signal sequences. For example, operative association of DNA with a promoter refers to the physical and functional relationship between the DNA and promoter such that the transcription of such DNA is initiated from the promoter by an RNA polymerase that specifically recognizes, binds to and transcribes the DNA. Thus, the term “operatively associated with” means that a nucleic acid construct containing the respective DNA sequences (represented by the terms “response element” and “non-endogenous or endogenous gene”) may contain additional nucleotide sequences (i.e., regulatory elements) that perform regulatory and effector functions controlling expression of a target gene.

Such regulatory elements, including response elements, are operatively associated with a suitable promoter for transcription of non-endogenous nucleic acid(s) product(s). As used herein, the term “promoter” refers to a specific nucleotide sequence recognized by RNA polymerase, the enzyme that initiates RNA synthesis. The promoter sequence is the site at which transcription can be specifically initiated under proper conditions. When non-endogenous nucleic acid(s), operatively associated with a suitable promoter, is(are) introduced into the cells of a suitable host, expression of the non-endogenous nucleic acid(s) is(are) controlled in many, but not all cases, by the presence of ligands, which are not normally present in the host cells.

Promoters contemplated for controlling expression of non-endogenous nucleic acids employed in the practice of the present invention include inducible (e.g., minimal CMV promoter, minimal TK promoter, modified MMLV LTR), constitutive (e.g., chicken β-actin promoter, MMLV LTR (non-modified), DHFR), and/or tissue specific promoters.

Inducible promoters contemplated for use in the practice of the present invention comprise transcription regulatory regions that function maximally to promote transcription of mRNA under inducing conditions. Examples of suitable inducible promoters include DNA sequences corresponding to: the E. coli lac operator responsive to IPTG (see Nakamura et al., Cell, 18:1109-1117, 1979); the metallothionein promoter metal-regulatory-elements responsive to heavy-metal (e.g., zinc) induction (see Evans et al., U.S. Pat. No. 4,870,009), the phage T71ac promoter responsive to IPTG (see Studier et al., Meth. Enzymol., 185: 60-89, 1990; and U.S. Pat. No. 4,952,496), the heat-shock promoter; the TK minimal promoter; the CMV minimal promoter; a synthetic promoter; and the like.

Exemplary constitutive promoters contemplated for use in the practice of the present invention include the CMV promoter, the SV40 promoter, the DHFR promoter, the mouse mammary tumor virus (MMTV) steroid-inducible promoter, Moloney murine leukemia virus (MMLV) promoter, elongation factor 1a (EF1a) promoter, albumin promoter, APO A1 promoter, cyclic AMP dependent kinase II (CaMKII) promoter, keratin promoter, CD3 promoter, immunoglobulin light or heavy chain promoters, neurofiliment promoter, neuron specific enolase promoter, L7 promoter, CD2 promoter, myosin light chain kinase promoter, HOX gene promoter, thymidine kinase (TK) promoter, RNA Pol II promoter, MYOD promoter, MYF5 promoter, phosphoglycerokinase (PGK) promoter, Stf1 promoter, Low Density Lipoprotein (LDL) promoter, chicken b-actin promoter (used in conjunction with ecdysone response element), and the like.

As readily understood by those of skill in the art, the term “tissue specific” refers to the substantially exclusive initiation of transcription in the tissue from which a particular promoter that drives gene expression is derived (e.g., expressed only in T-cells, endothelial cells, smooth muscle cells, and the like). Exemplary tissue specific promoters contemplated for use in the practice of the present invention include the GH promoter, the NSE promoter, the GFAP promoter, neurotransmitter promoters (e.g., tyrosine hydroxylase, TH, choline acetyltransferase, CHAT, and the like), promoters for neurotropic factors (e.g., a nerve growth factor promoter, NT-3, BDNF promoters, and the like), and so on.

As used herein, when referring to nucleic acids, the phrase “non-endogenous to said host” or simply “non-endogenous” refers to nucleic acids not naturally found at levels sufficient to provide a function in the particular cell where transcription is desired. For example, non-endogenous nucleic acids can be either natural or synthetic nucleic acids, which are introduced into the host in the form of DNA or RNA. The nucleic acids of interest can be introduced into target cells (for in vitro applications), or the nucleic acids of interest can be introduced directly or indirectly into a host, for example, by the transfer of transformed cells into a host.

In contrast to non-endogenous nucleic acids, the phrase “endogenous nucleic acids” or “endogenous genes” refers to nucleic acids naturally found at levels sufficient to provide a function in the particular cell where transcription is desired.

Non-endogenous nucleic acids contemplated for use in the practice of the present invention include wild type and/or therapeutic nucleic acids. “Wild type” genes are those that are native to cells of a particular type. Exemplary wild type nucleic acids are genes which encode products the substantial absence of which leads to the occurrence of a non-normal state in a host; or a substantial excess of which leads to the occurrence of a non-normal state in a host.

Such genes may not be expressed in biologically significant levels or may be undesirably overexpressed. Thus, for example, while a synthetic or natural gene coding for human insulin would be non-endogenous genetic material to a yeast cell (since yeast cells do not naturally contain insulin genes), a human insulin gene inserted into a human skin fibroblast cell would be a wild type gene with respect to the fibroblast since human skin fibroblasts contain genetic material encoding human insulin, although human skin fibroblasts do not express human insulin in biologically significant levels.

Therapeutic nucleic acids contemplated for use in the practice of the present invention include those which encode products which are toxic to the cells in which they are expressed; or encode products which impart a beneficial property to a host; or those which transcribe nucleic acids which modulate transcription and/or translation of endogenous genes.

As employed herein, the phrase “therapeutic nucleic acids” refers to nucleic acids that impart a beneficial function to the host in which such nucleic acids are transcribed. Therapeutic nucleic acids are those that are not naturally found in host cells. For example, synthetic or natural nucleic acids coding for wild type human insulin would be therapeutic when inserted into a skin fibroblast cell so as to be expressed in a human host, where the human host is not otherwise capable of expressing functionally active human insulin in biologically significant levels. Further examples of therapeutic nucleic acids include nucleic acids that transcribe antisense constructs used to suppress the expression of endogenous genes. Such antisense transcripts bind endogenous nucleic acid (mRNA or DNA) and effectively cancel out the expression of the gene. In accordance with the methods described herein, therapeutic nucleic acids are expressed at a level that provides a therapeutically effective amount of the corresponding therapeutic protein.

Non-endogenous nucleic acids useful in the practice of the present invention include genes that encode biologically active proteins of interest, such as, e.g., secretory proteins that can be released from a cell; enzymes that can metabolize a toxic substance to produce a non-toxic substance, or that metabolize an inactive substance to produce a useful substance; regulatory proteins; cell surface receptors; and the like. Useful genes include genes that encode blood clotting factors, such as human factors VIII and IX; genes that encode hormones, such as insulin, parathyroid hormone, luteinizing hormone releasing factor (LHRH), alpha and beta seminal inhibins, and human growth hormone; genes that encode proteins, such as enzymes, the absence of which leads to the occurrence of an abnormal state; genes encoding cytokines or lymphokines such as interferons, granulocytic macrophage colony stimulating factor (GM-CSF), colony stimulating factor-1 (CSF-1), tumor necrosis factor (TNF), and erythropoietin (EPO); genes encoding inhibitor substances such as alpha₁-antitrypsin; genes encoding substances that function as drugs, e.g., genes encoding the diphtheria and cholera toxins; and the like.

Additional nucleic acids contemplated for use in accordance with the present invention include genes that encode proteins present in dopaminergic neurons (useful, for example, for the treatment of Parkinson's disease), cholinergic neurons (useful, for example, for the treatment of Alzheimer's disease), hippocampal pyramidal neurons (also useful for the treatment of Alzheimer's disease), norepinephrine neurons (useful, for example, for the treatment of epilepsy), spinal neurons (useful, for example, for the treatment of spinal injury), glutamatergic neurons (useful, for example, for the treatment of schizophrenia), cortical neurons (useful, for example, for the treatment of stroke and brain injury), motor and sensory neurons (useful, for example, for the treatment of amyotrophic lateral sclerosis), and the like.

Typically, nucleic acid sequence information for proteins encoded by non-endogenous nucleic acid(s) contemplated for use herein can be located in one of many public access databases, e.g., GENBANK, EMBL, Swiss-Prot, and PIR, or in related journal publications. Thus, those of skill in the art have access to sequence information for virtually all known genes. Those of skill in the art can obtain the corresponding nucleic acid molecule directly from a public depository or from the institution that published the sequence. Optionally, once the nucleic acid sequence encoding a desired protein has been ascertained, the skilled artisan can employ routine methods, e.g., polymerase chain reaction (PCR) amplification, to isolate the desired nucleic acid molecule from the appropriate nucleic acid library. Thus, all known nucleic acids encoding proteins of interest are available for use in the methods and products described herein.

Additional components that can optionally be incorporated into the invention constructs include selectable marker genes and genes encoding proteins required for retroviral packaging, e.g., the pol gene, the gag gene, the env gene, and the like.

Selectable marker genes contemplated for use in the practice of the present invention include antibiotic resistance genes, genes that enable cells to process metabolic intermediaries, and the like. Exemplary antibiotic resistance genes include genes which impart tetracycline resistance, genes that impart ampicillin resistance, neomycin resistance, hygromycin resistance, puromycin resistance, and the like.

Genes that enable cells to process metabolic intermediaries include genes which permit cells to incorporate L-histidinol, genes encoding thymidine kinase, genes encoding xanthine-guanine phosphoribosyl transferase (gpt), genes encoding dihydrofolate reductase, genes encoding asparagine synthetase, and the like.

As employed herein, the terms “subject,” “subject organism” and “host” refer to the cell, tissue, organ or organism in need of transcriptional regulation of non-endogenous or endogenous nucleic acids. The subject organism can be mammalian or mammalian-derived cells or tissue. Exemplary mammals include: humans; domesticated animals, e.g., rat, mouse, rabbit, canine, feline, and the like; farm animals, e.g., chicken, bovine, ovine, porcine, and the like; animals of zoological interest, e.g., monkey, baboon, and the like, or a cell thereof. Alternatively, a subject organism can be non-mammalian, preferably non-insect, such as yeast, plant, fungus, bacterial, or other non-mammalian species, or a cell of such a non-mammalian species.

In accordance with one aspect of the present invention, unless and until a suitable ligand is administered to the host, substantially no transcription of the desired non-endogenous nucleic acids occurs. Since ecdysteroids, for example, are not naturally present in mammalian, plant and fungal systems, and the like, if it is desired that transcription of a particular non-endogenous nucleic acid be under precise control of the practitioner, a first chimeric protein containing a DNA binding domain and/or dimerization domain derived from an ecdysone receptor and a second chimeric protein that is a suitable partner for associating therewith, as taught herein, is used and the non-endogenous nucleic acid is put under the control of an ecdysone response element, i.e. a response element to which an activated ecdysone receptor binds in nature.

As employed herein, the term “ligand” (or ligand precursor) refers to a steroidal or non-steroidal substance or compound which, in its native state (or after conversion to its “active” form), binds to at least one of the chimeric proteins, or to the dimerized invention system, thereby creating a ligand/dimer complex, which in turn can bind an appropriate response element and activate transcription therefrom. Ligands function to modulate transcription of nucleic acid(s) maintained under the control of a response element. Such ligands are well known in the art and include synthetic organic compounds, e.g., quinolines, isoquinolines (e.g., dihydroxy isoquinoline, indoloisoquinoline, and the like), hydrazines (e.g., diacyl hydrazines), plant derived steroids, insect derived steroids, synthetic steroids, and the like.

The terms “ecdysone” and “ecdysteroid” as interchangeably used herein, are employed in the generic sense (in accordance with common usage in the art), referring to a family of ligands with the appropriate binding and transactivation activity (see, for example, Cherbas et al., in Biosynthesis, metabolism and mode of action of invertebrate hormones (Ed. J. Hoffmann and M. Porchet), Springer-Verlag, Berlin, p 305-322. An ecdysone, therefore, is a compound which acts to modulate gene transcription for a gene maintained under the control of an ecdysone response element.

20-Hydroxy-ecdysone (also known as β-ecdysone) is the major naturally occurring ecdysone (found, for example, in plants). Unsubstituted ecdysone (also known as α-ecdysone) is converted in peripheral tissues to β-ecdysone. Analogs of the naturally occurring ecdysones are also contemplated within the scope of the present invention. Examples of such analogs, commonly referred to as ecdysteroids, include ponasterone A, 26 iodoponasterone A, muristerone A, inokosterone, 26-mesylinokosterone, and the like. Since it has been previously reported that the above-described ecdysones are not toxic, teratogenic, or known to affect mammalian physiology, they are ideal candidates for use as inducers in cultured cells and transgenic mammals according to the invention methods.

Other phytoecdysteroids are also contemplated for use in the practice of the invention as ligands of invention systems that recognize ecdysone response elements. Such phytoecdysteroids are known in the art (J. H. Adler et al., Lipids 30(3):257-62, 1995). The biological effect of phytoecdysteroids in higher animals are also known (V. N. Syrov, Eksp. Klin. Farmakol. 57(5):61-6, 1994).

Non-steroidal ligands are also contemplated for use in the practice of the present invention. For example, when a ligand not normally present in the cells of the host to be treated is desired (i.e., a ligand non-endogenous to the host), a synthetic organic compound such as a hydrazine can be employed as the ligand. Hydrazines are presently preferred as they include compounds that are readily available and/or are relatively inexpensive to manufacture. One such compound, tebufenozide, is a non-steroidal ecdysone agonist which is commercially available. This compound specifically targets lepidopteran species, including Bombyx mori. Tebufenozide has undergone extensive testing in animal hosts and has proved to be of very low toxicity to mammals and other non-insect species.

Additional exemplary hydrazines contemplated for use herein are well known in the art, including those disclosed in U.S. patent application Ser. No. 09/005,286, filed Jan. 9, 1998, which is incorporated herein by reference in its entirety. Exemplary hydrazines include 1,2-diacyl hydrazines (e.g., tebufenozide), N′-substituted-N,N′-disubstituted hydrazines, dibenzoylalkyl cyanohydrazines, N-substituted-N-alkyl-N,N-diaroyl hydrazines, N-substituted-N-acyl-N-alkyls, carbonyl hydrazines, N-aroyl-N′-alkyl-N′-aroyl hydrazines, and the like. Since it has been previously reported that the above-described diacyl hydrazines are neither toxic, teratogenic, nor known to affect mammalian physiology, they are ideal candidates for use as exogenous ligands (e.g. as inducers) in cultured cells and transgenic mammals according to invention methods.

Ligands, and formulations containing them, are administered in a manner compatible with the desired route of administration, the dosage formulation, and in a therapeutically effective amount. The required dosage will vary with the particular treatment desired, the degree and duration of therapeutic effect desired, the judgment of the practitioner, as well as properties peculiar to each individual. Moreover, suitable dosage ranges for systemic application depend on the route of administration. It is anticipated that dosages between about 10 micrograms and about 1 milligram per kilogram of body weight per day will be used for therapeutic treatment.

An effective amount of ligand contemplated for use in the practice of the present invention is the amount of ligand required to achieve the desired level of transcription and/or translation of non-endogenous nucleic acid. A therapeutically effective amount is typically an amount of ligand or ligand precursor that, when administered in a physiologically acceptable composition, is sufficient to achieve a plasma concentration of the active ligand from about 0.1 mg/ml to about 100 mg/ml, for example, from about 1.0 mg/ml to about 50 mg/ml, and preferably at least about 2 mg/ml and usually 5 to 10 mg/ml.

Ligand can be administered in a variety of ways, as are well-known in the art, i.e., by any means that produces contact between ligand and the invention system. For example, such ligands can be administered topically, orally, intravenously, intraperitoneally, intravascularly, and the like. The administration can be by any conventional means available for use in conjunction with pharmaceuticals, e.g., by intravenous injection, either as individual therapeutically active ingredients or in a combination with other therapeutically active ingredients. Ligands contemplated for use in the practice of the present invention can be administered alone, but are generally administered with a pharmaceutical carrier selected on the basis of the chosen route of administration and standard pharmaceutical practice.

In accordance with a particular embodiment of the present invention, pharmaceutically acceptable formulations, and kits, comprising at least one ligand for an invention functional entity, for example an ecdysteroid, such as ecdysone or 20-hydroxy ecdysone, and a pharmaceutically acceptable carrier are contemplated. In accordance with another aspect of the present invention, pharmaceutically acceptable formulations consisting essentially of at least one ligand and a pharmaceutically acceptable carrier, are contemplated. Pharmaceutical formulations of the present invention can be used in the form of a solid, a solution, an emulsion, a dispersion, a micelle, a liposome, and the like, wherein the resulting formulation contains one or more of the ligands of the present invention, as an active ingredient, in admixture with an organic or inorganic carrier or excipient suitable for enteral or parenteral applications.

The ligand(s) may be compounded, for example, with the usual non-toxic, pharmaceutically acceptable carriers suitable for administration by oral, topical, nasal, transdermal, intravenous, subcutaneous, intramuscular, intracutaneous, intraperitoneal, intravascular, and the like means. Administration in the form of creams, lotions, tablets, dispersible powders, granules, syrups, elixirs, sterile aqueous or non-aqueous solutions, suspensions or emulsions, and the like, is contemplated. Exemplary pharmaceutically acceptable carriers include carriers for tablets, pellets, capsules, suppositories, solutions, emulsions, suspensions, and any other form suitable for use. Such carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and/or perfumes may be used. The active compound (e.g., ecdysteroid as described herein) is included in the pharmaceutically acceptable formulation in an amount sufficient to produce the desired effect upon the process or condition of diseases.

Pharmaceutically acceptable formulations containing ligand(s) as active ingredient may be in a form suitable for oral use, for example, as aqueous or oily suspensions, syrups or elixirs, tablets, troches, lozenges, dispersible powders or granules, emulsions, or hard or soft capsules. For the preparation of oral liquids, suitable carriers include emulsions, solutions, suspensions, syrups, and the like, optionally containing additives such as wetting agents, emulsifying and suspending agents, dispersing agents, sweetening, flavoring, coloring, preserving and perfuming agents, and the like. Formulations intended for oral use may be prepared according to any method known to the art for the manufacture of pharmaceutically acceptable formulations.

Tablets containing ligand(s) as active ingredient in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874, to form osmotic therapeutic tablets for controlled release.

In some cases, formulations for oral use may be in the form of hard gelatin capsules wherein the ligand is mixed with an inert solid diluent, for example, calcium carbonate, calcium phosphate, or kaolin. They may also be in the form of soft gelatin capsules wherein the ligand is mixed with water or an oil medium, for example, peanut oil, liquid paraffin, or olive oil.

The pharmaceutically acceptable formulations may be in the form of a sterile injectable suspension. Suitable carriers include non-toxic parenterally-acceptable sterile aqueous or non-aqueous solutions, suspensions, or emulsions. This suspension may be formulated according to known methods using suitable dispersing or wetting agents and suspending agents. They can also be manufactured in the form of sterile water, or some other sterile injectable medium immediately before use. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. They may be sterilized, for example, by filtration through a bacteria-retaining filter, by incorporating sterilizing agents into the formulations, by irradiating the formulations, or by heating the formulations. Sterile injectable suspensions may also contain adjuvants such as preserving, wetting, emulsifying, and dispersing agents. Buffers, preservatives, antioxidants, and the like can be incorporated as required.

Compounds contemplated for use in the practice of the present invention may also be administered in the form of suppositories for rectal administration of the drug. These formulations may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

Pharmaceutically acceptable formulations containing suitable ligand(s) are preferably administered intravenously, for example, as by injection of a unit dose. The term “unit dose,” when used in reference to a pharmaceutically acceptable formulation of the present invention, refers to a quantity of the pharmaceutical formulation suitable as unitary dosage for the subject, each unit containing a predetermined quantity of active material calculated to produce the desired therapeutic effect in association with the required diluent, i.e., carrier, or vehicle. It may be particularly advantageous to administer such formulations in depot or long-lasting form as discussed hereinafter.

Therapeutic compositions or pharmaceutically acceptable formulations containing suitable ligand are preferably administered intravenously, as by injection of a unit dose, for example. The term “unit dose,” when used in reference to a therapeutic composition of the present invention, refers to a quantity of ligand suitable as unitary dosage for the subject, each unit containing a predetermined quantity of active material calculated to produce the desired therapeutic effect in association with the required diluent, i.e., carrier, or vehicle. It may be particularly advantageous to administer such compounds in depot or long-lasting form.

Suitable regimes for initial administration and booster shots are variable, but are typified by an initial administration followed by repeated doses at one or more intervals, by a subsequent injection, or other administration. Alternatively, continuous intravenous infusion sufficient to maintain concentrations in the blood in the ranges specified for in vivo therapies are contemplated.

In accordance with another embodiment of the present invention, there are provided methods for producing transgenic animals capable of prolonged and regulated expression of non-endogenous nucleic acid(s). The invention method for producing a transgenic animal comprises introducing into early-stage embryos or stem cells of the animal:

(i) a nucleic acid construct comprising a promoter and said non-endogenous nucleic acid(s) under the control of a response element; and

(ii) nucleic acid encoding the invention system comprising first and second chimeric proteins, wherein a functional entity formed by the chimeric proteins activates or represses the response element in the presence of a ligand.

As used herein, the phrase “transgenic animal” refers to an animal that contains one or more expression constructs containing one or more non-endogenous nucleic acid(s) under the transcription control of an operator and/or response element as described herein.

Methods of making transgenic animals using a particular nucleic acid construct are well-known in the art. When preparing invention transgenic animals, it is presently preferred that two transgenic lines are generated. The first line will express, for example, the first and second chimeric proteins as described above. Tissue specificity is conferred by the selection of a tissue-specific promoter (e.g., T-cell specific) that will direct expression of the chimeric protein to appropriate tissue. A second line contains a nucleic acid construct comprising a promoter and non-endogenous nucleic acid under the control of a response element, for example, an endogenous response element. Crossbreeding of these two lines will provide transgenic animal(s) that expresses an invention system and the non-endogenous nucleic acid.

In a presently preferred embodiment, an invention transgenic animal contains one or more expression constructs containing nucleic acid encoding the invention first and second chimeric proteins and non-endogenous nucleic acid under the transcription control of a response element. Thus, with tissue specific expression of the chimeric proteins as described above and timely ligand treatment, gene expression can be induced or repressed with spatial, dosage, and/or temporal specificity.

In accordance with yet another embodiment of the present invention, there are provided methods for modulating the transcription of one or more target nucleic acid(s) in a subject containing:

(i) a nucleic acid construct comprising a promoter and said target nucleic acid(s) under the control of a response element; and

(ii) nucleic acid(s) under the control of an inducible promoter, said nucleic acid(s) encoding an invention system comprising first and second chimeric proteins wherein the functional entity formed by the invention chimeric proteins activates or represses the response element in the presence of a ligand for the dimer;

said method comprising introducing a ligand not normally present in the cells of the subject and subjecting the subject to conditions suitable to induce or repress expression of the invention system.

In accordance with yet another embodiment of the present invention, there are provided methods for the expression of recombinant products detrimental to a subject organism, said method comprising:

(a) transforming suitable cells in the organism with:

(i) a nucleic acid construct comprising a promoter and non-endogenous nucleic acid(s) which express the recombinant product under the control of a response element that is not normally present in the cells of said organism, and

(ii) nucleic acid encoding the invention system,

wherein the invention chimeric proteins activate the regulatory element in the presence of a ligand for the functional entity;

(b) growing said cells to the desired level in the substantial absence of the ligand; and

(c) inducing expression of said recombinant product by administering to the organism a ligand, which, in combination with said dimer, binds to said response element and activates transcription therefrom.

Modified cells are cultivated under growth conditions (as opposed to protein expression conditions) until a desired density is achieved. For example, stably transfected mammalian cells may be prepared by transfecting cells with an expression vector having a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a non-endogenous reporter gene (such as the E. coli β-galactosidase gene) to monitor transfection efficiency. Selectable marker genes are typically not included in the transient transfections because the transfectants are typically not grown under selective conditions, and are usually analyzed within a few days after transfection.

Recombinant products detrimental to a host organism contemplated for expression in accordance with the present invention include any gene product that functions to confer a toxic effect on the organism. For example, tissue specific inducible expression of a toxin, such as the diphtheria toxin, would allow for specific ablation of tissue (Ross et al. Genes and Development 7:1318-1324 (1993)), for example to create a new phenotype in the transgenic animal. Moreover, the numerous gene products known to induce apoptosis in cells expressing such products are contemplated for use herein (see, e.g, Apoptosis, The Molecular Basis of Cell Death, Current Communications In Cell & Molecular Biology, Cold Spring Harbor Laboratory Press, 1991).

In accordance with still another embodiment of the present invention, there are provided methods for the controlled expression of recombinant products required for the growth and maintenance of a subject organism, said method comprising:

(a) transforming suitable cells in the organism with:

(i) a nucleic acid construct comprising a promoter and non-endogenous nucleic acid(s) which express the recombinant product under the control of a response element that is not normally present in the cells of said organism, and

(ii) nucleic acid encoding the invention system,

wherein the invention chimeric proteins activate the regulatory element in the presence of a ligand for the functional entity;

(b) growing said cells to the desired level in the presence of the ligand; which, in combination with said dimer, binds to said response element and promotes transcription therefrom, and

(c) terminating expression of said recombinant product by discontinuing administration of ligand to the organism.

In accordance with this aspect of the present invention, modified cells (or host animals containing such cells) are cultivated in the presence of ligand until a desired stage of development is achieved, then administration of ligand is discontinued. Absence of ligand causes expression of the recombinant product to cease. Thus, in accordance with this aspect of the present invention, inducible expression of proteins necessary for development is provided as needed during development, then discontinued at a desired stage of development, thereby providing knock-out models of isolated cells and animals containing same, wherein the subject cell or animal would not survive if expression of the “deleted” protein were prematurely terminated.

In accordance with yet another embodiment of the present invention, there are provided methods for the treatment of a host in need of gene therapy, said method comprising:

introducing into cells of said host:

(i) nucleic acid encoding an invention system as described herein; and

(ii) a nucleic acid construct comprising a promoter and nucleic acid(s) encoding therapeutic protein(s) under the control of a response element responsive to the DNA binding domain in the invention system,

wherein the functional entity formed by association of the invention chimeric proteins activates or represses the response element in the presence of a ligand for the functional entity, and

administering, to said host, an effective amount of ligand for the invention system.

Optionally, the cells can be obtained from the host, modified as above, and then reintroduced into the host organism. For example, the non-endogenous nucleic acid can be introduced directly into cells obtained from a donor (host or separate donor) and the modified cells then can be implanted within the host organism. In a presently preferred embodiment, the transplanted cells are autologous with respect to the host. “Autologous” means that the donor and recipient of the cells are one and the same.

Cells can be modified by “in vivo delivery” of biological materials by such routes of administration as oral, intravenous, subcutaneous, intraperitoneal, intrathecal, intramuscular, intracranial, inhalational, topical, transdermal, suppository (rectal), pessary (vaginal), and the like. The non-endogenous nucleic acid may be stably incorporated into cells or may be transiently expressed using methods known in the art.

The concept of gene replacement therapy for humans involves the introduction of functionally active “wild type” or “therapeutic” nucleic acids into the somatic cells of an affected host to correct a gene defect or deficiency. However, in order for gene replacement therapy to be effective, it must be possible to control the time and location at which gene expression occurs.

Genes that encode useful “gene therapy” proteins that are not normally transported outside the cell can be used in the invention if such genes are “functionally appended” to, or operatively associated with, a signal sequence that can “transport” the encoded product across the cell membrane. A variety of such signal sequences are known and can be used by those skilled in the art without undue experimentation.

Gene transfer vectors (also referred to as “expression vectors”) contemplated for use herein are recombinant nucleic acid molecules that are used to transport nucleic acid into host cells for expression and/or replication thereof. Expression vectors may be either circular or linear, and are capable of incorporating a variety of nucleic acid constructs therein. Expression vectors typically come in the form of a plasmid that, upon introduction into an appropriate host cell, results in expression of the inserted nucleic acid.

Suitable expression vectors for use herein are well known to those of skill in the art and include recombinant DNA or RNA construct(s), such as plasmids, phage, recombinant virus or other vectors that, upon introduction into an appropriate host cell, result(s) in expression of the inserted DNA. Appropriate expression vectors are well known to those of skill in the art and include those that are replicable in eukaryotic cells and/or prokaryotic cells and those that remain episomal or those which integrate into the host cell genome. Expression vectors typically further contain other functionally important nucleic acid sequences encoding antibiotic resistance proteins, and the like.

The amount of non-endogenous nucleic acid introduced into a host organism, cell or cellular system can be varied by those of skill in the art. For example, when a viral vector is employed to achieve gene transfer, the amount of nucleic acid introduced can be varied by varying the amount of plaque forming units (PFU) of the viral vector.

As used herein, the phrase “transcription regulatory region” refers to that portion of a nucleic acid or gene construct that controls the initiation of mRNA transcription. Regulatory regions contemplated for use herein, in the absence of the non-mammalian transactivator, typically comprise at least a minimal promoter in combination with a regulatory element responsive to the complex of the invention system with the ligand. A minimal promoter, when combined with a regulatory element, functions to initiate mRNA transcription in response to such a complex. However, transcription will not occur unless the required inducer (ligand therefor) is present. However, as described herein, certain of the invention systems activate or repress mRNA transcription even in the absence of ligand for the invention system.

Preferably, the transcription regulatory region further comprises a binding site for ubiquitous transcription factor(s). Such binding sites are preferably positioned between the promoter and the regulatory element. Suitable ubiquitous transcription factors for use herein are well-known in the art and include, for example, Sp1.

Exemplary eukaryotic expression vectors include eukaryotic constructs, such as the pSV-2 gpt system (Mulligan et al., (1979) Nature, 277:108-114); pBlueSkript (Stratagene, La Jolla, Calif.), the expression cloning vector described by Genetics Institute (Science, (1985) 228:810-815), and the like. Each of these plasmid vectors is capable of promoting expression of the chimeric proteins of interest.

Suitable means for introducing (transducing) expression vectors containing invention nucleic acid constructs into host cells to produce transduced recombinant cells (i.e., cells containing recombinant heterologous nucleic acid) are well-known in the art (see, for review, Friedmann, Science, 244:1275-1281, 1989; Mulligan, Science, 260:926-932. 1993, each of which are incorporated herein by reference in their entirety). Exemplary methods of transduction include, e.g., infection employing viral vectors (see, e.g., U.S. Pat. No. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The transduced nucleic acid can optionally include sequences which allow for its extrachromosomal (i.e., episomal) maintenance, or the transduced nucleic acid can be donor nucleic acid that integrates into the genome of the host.

In a specific embodiment, a gene transfer vector contemplated for use herein is a viral vector, such as Adenovirus, adeno-associated virus, a herpes-simplex virus based vector, a synthetic vector for gene therapy, and the like (see, e.g., Suhr et al., Arch. of Neurol. 50:1252-1268, 1993). Preferably, a gene transfer vector employed herein is a retroviral vector. Retroviral vectors contemplated for use herein are gene transfer plasmids that have an expression construct containing an non-endogenous nucleic acid residing between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764).

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, each of which is hereby incorporated herein by reference, in its entirety. These documents provide a description of methods for efficiently introducing nucleic acids into human cells using such retroviral vectors. Other retroviral vectors include, for example, mouse mammary tumor virus vectors (e.g., Shackleford et al., (1988) PNAS, USA, 85:9655-9659), human immunodeficiency virus (e.g., Naldini et al. (1996) Science 272:165-320), and the like.

Various procedures are also well-known in the art for providing helper cells which produce retroviral vector particles that are essentially free of replicating virus. See, for example, U.S. Pat. No. 4,650,764; Miller, Human Gene Therapy, 1:5-14, 1990; Markowitz, et al., Journal of Virology, 61(4):1120-1124, 1988; Watanabe, et al., Molecular and Cellular Biology, 3(12):2241-2249, 1983; Danos, et al., PNAS, 85:6460-6464, 1988; and Bosselman, et al., Molecular and Cellular Biology, 72(5):1797-1806, 1987, which disclose procedures for producing viral vectors and helper cells that minimize the chances for producing a viral vector that includes a replicating virus.

Recombinant retroviruses suitable for carrying out the invention methods are produced employing well-known methods for producing retroviral virions. See, for example, U.S. Pat. No. 4,650,764; Miller, supra 1990; Markowitz, et al., supra 1988; Watanabe, et al., supra 1983; Danos, et al., PNAS, 85:6460-6464, 1988; and Bosselman, et al., Molecular and Cellular Biology, 7(5):1797-1806, 1987.

For example, in one embodiment, a modular assembly retroviral vector (MARV) can be utilized to express both the first and second invention chimeric proteins and an antibiotic resistance gene. A “covector” (referred to herein as MARSHA) can be utilized to provide a nucleic acid construct comprising the promoter, the regulatory element and non-endogenous nucleic acid, and a second antibiotic resistance gene. The MARSHA vector carrying non-endogenous nucleic acid also has LTRs modified to promote high-level expression only in the presence of the invention chimeric protein encoded by MARV and non-endogenous ligand therefor. Co-infected primary mammalian cells can then be selected using both antibiotics, resulting in a cell population that is dependent on ligand for high-level expression of the non-endogenous nucleic acid.

By introducing all of the necessary regulatory machinery, plus non-endogenous nucleic acid, selectable markers, and nucleic acid encoding invention system, e.g., into a MARV retrovirus, highly efficient insertion of non-endogenous nucleic acids into targeted cells can be achieved.

The simultaneous preparation of the invention first and second chimeric proteins chains by co-expression is presently preferred. In nature, the association of different subunits of complex proteins takes place after protein synthesis. Other components of the cellular apparatus frequently participate in this association as catalysts or controlling elements, with folding of the original structures taking place on occasion. Disturbances of the association, e.g. by an equal synthesis of the individual components, can have negative consequences both for the proteins which are to be formed and for the host cell. In nature, this system is subject to sophisticated regulation, which is for the most part cell-specific. Since this regulation is in general not adjustable in genetically manipulated cells, the alternatives explained below have been developed and used for the simultaneous preparation of several foreign proteins:

1) The genes separately encoding the first and second chimeric proteins can be integrated separately into expression vectors and then cotransferred in an appropriate ratio into the cells. This presupposes that several plasmid copies are taken up at the same time in a stable manner and continue to be harbored during division. The ratio of the expression of the different genes to each other depends both on the copy number and on the site of integration in the genome of the host cell. It is possible, by elaborate screening processes, to isolate cell clones which express the individual gene products in the desired ratio.

2) In order to level out the copy number, the different genes can be placed in independent transcription units on one vector. While this, to a large extent, ensures stoichiometric representation of the genes, this process is also subject to problems. Thus, even if expression units having promoters of equal strength are used, it is in no way guaranteed that the mRNAs, which encode the different proteins, have the same stability and translation efficiency. Nor does the transcriptional efficiency of the two genes necessarily need to be identical. In this case, the stoichiometry of expression is produced step-wise using recombinant DNA stratagems (positioning of the transcription units with respect to each other and modulation of the strength of the individual promoters by removing or adding individual elements).

3) Bicistronic or multicistronic vectors have been developed in order to avoid the problems connected with the stability of the mRNA of different transcripts. For this purpose, the individual reading frames of the gene segments—cistrons—encoding the chimeric proteins lie on one transcription unit (expression unit). Expression of the multicistronic gene is effected using a single promoter. While the first cistron in such vectors is normally translated very efficiently, translation of the subsequent cistrons depends on the intercistronic sequences. If normal 5′ untranslated sequences (5′UTR) from monocistronic genes are used for these intercistronic sequences, expression of the subsequent cistron is usually very low (as a rule, about 0.5 to 2% of the translation of the first cistron, R. J. Kaufman et al., EMBO J. 6:187-193, 1987; E. Boel et al., FEBS Lett. 219:181-188, 1987). It is possible to increase this efficiency to about 20% by inserting leader sequences (high efficiency leaders, HEL). By use of particular cellular and viral sequences which render possible internal initiation of translation (IRES; R. J. Jackson et al., Trends Biochem. Sci. 15:477-483, 1990), a translation ratio between the first and subsequent cistron of 3:1 can be achieved.

It is possible to improve the translational efficiency of the subsequent cistrons employing a variety of methods known in the art, for example by using so-called “high efficiency leader” sequences (HEL). However, even in constructs of this nature, the expression values which can be achieved for the second and subsequent cistrons are always clearly lower than those of the first cistron regulated in a “cap”-dependent manner.

Therefore, the mechanism presently preferred for initiation of translation internally makes use of specific nucleic acid sequences known as IRES. These sequences are known in the art and include the untranslated regions of individual picorna viruses, e.g. poliovirus and encephalomyocarditis virus as well as some cellular proteins, e.g. BiP. In the picorna viruses, a short segment of the 5′ untranslated region, the so-called the internal ribosomal entry site (IRES), is responsible for the internal binding of a preinitiation complex. In addition to this, further segments from this region are necessary for efficiently initiating this translation. Thus, it is evident, for example, that not only the 400 base pairs upstream of the IRES, but also the extreme 5′ part of the picorna virus untranslated region, are necessary for efficient translation (E. A. F. Simoes and P. Sarnow, J. Virol. 65:913-921, 1991). If the IRES is responsible for initiating the second cistron, that is if a cistron is situated between the “cap” and the IRES, the IRES elements can function as initiators of the efficient translation of reading frames while having no influence on the “cap”-dependent translation of the first cistron.

The invention will now be described in greater detail with reference to the following non-limiting examples.

### EXAMPLES

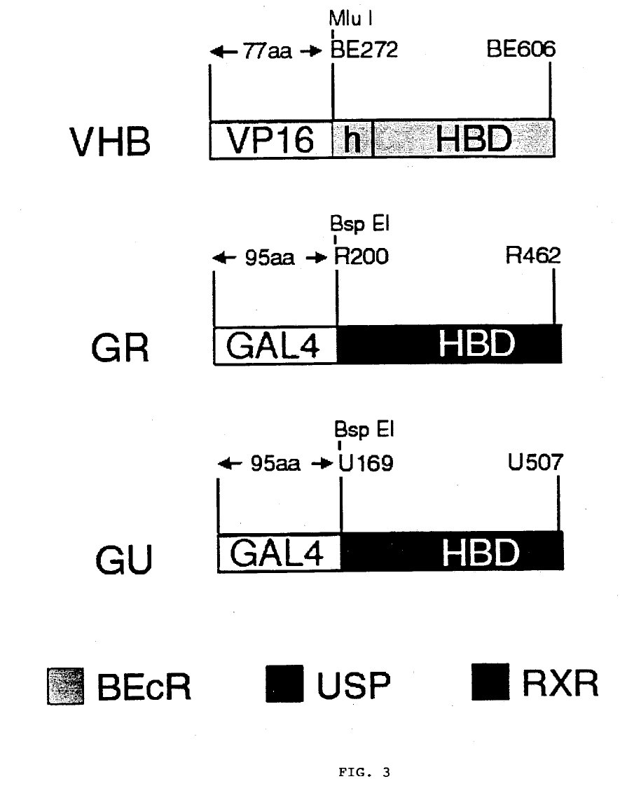

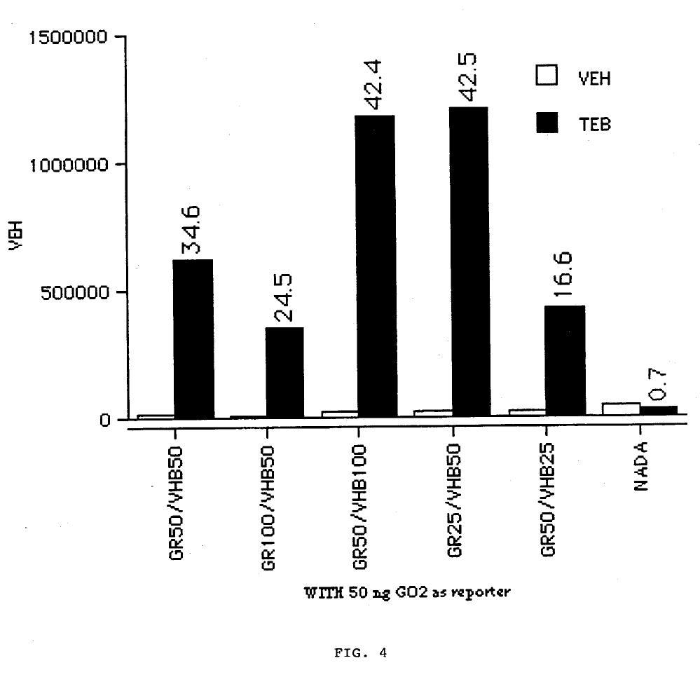

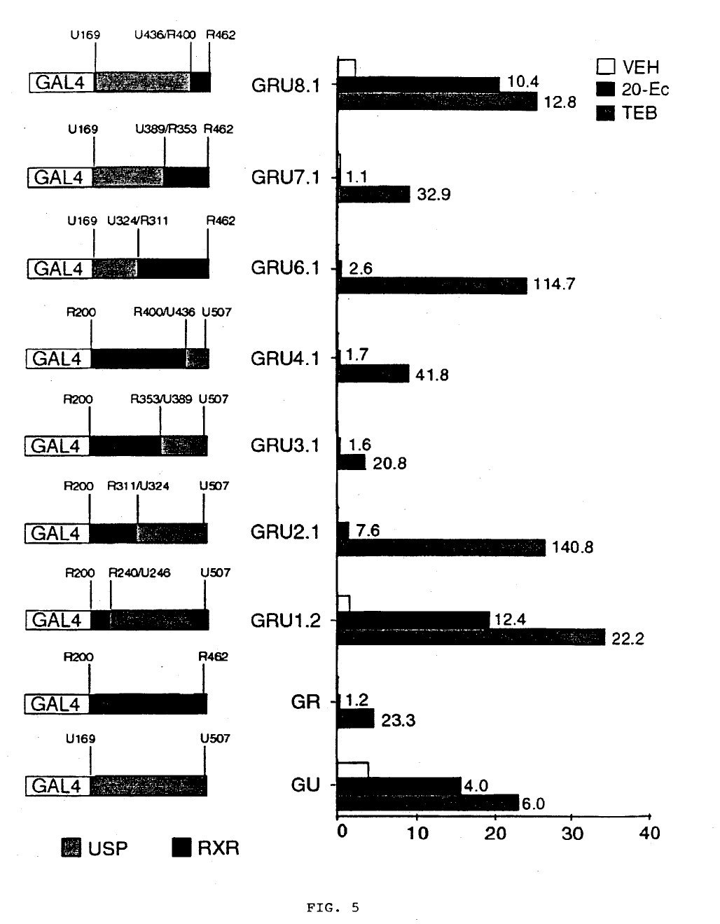

All construction and cloning was performed by standard methods and as described by Suhr et al., in Proc Natl Acad Sci USA 95(14):7999-8004 (1998) for similar chimeric proteins. For the VHB protein combining the transactivation domain of HSV VP16 protein with the hinge and hormone binding/heterodimerization domain (HBD) of Bombyx mori EcR (BEcR), the 77 amino acid (231 base pair) VP16 domain was amplified by PCR with an in-frame Mlul site at the 3′ end that was ligated with an identical site introduced into the 5′ end of the BEcR hinge domain and HBD (corresponding to amino acids 272 to 606). The final construct, termed VHB, functionally couples the VP16 domain with the BEcR high-affinity hormone responsive domains (see FIG. 3). This DNA was introduced into a number of expression constructs for expression in transduced cells.

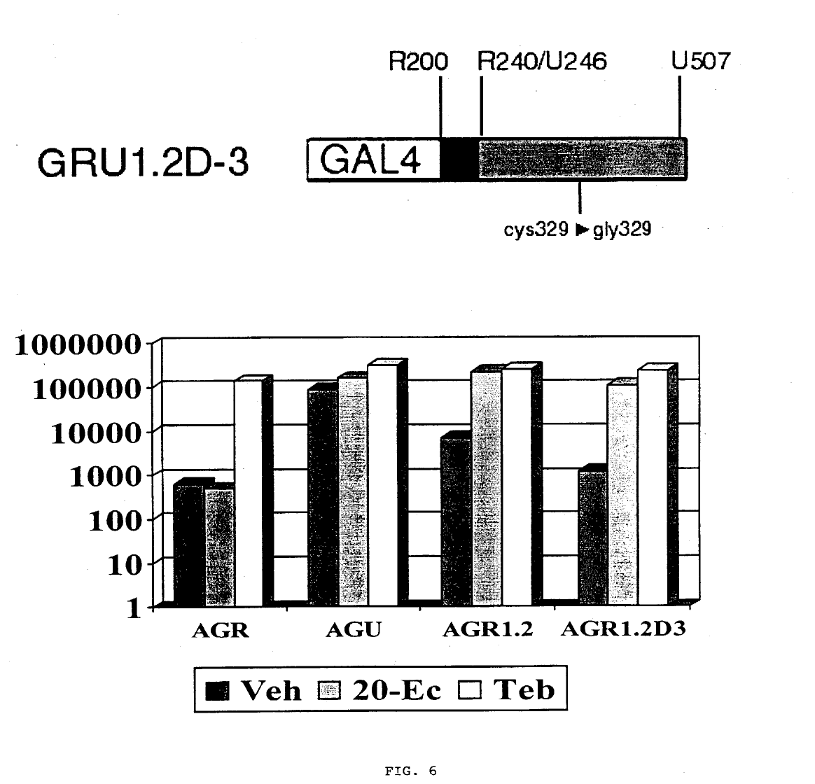

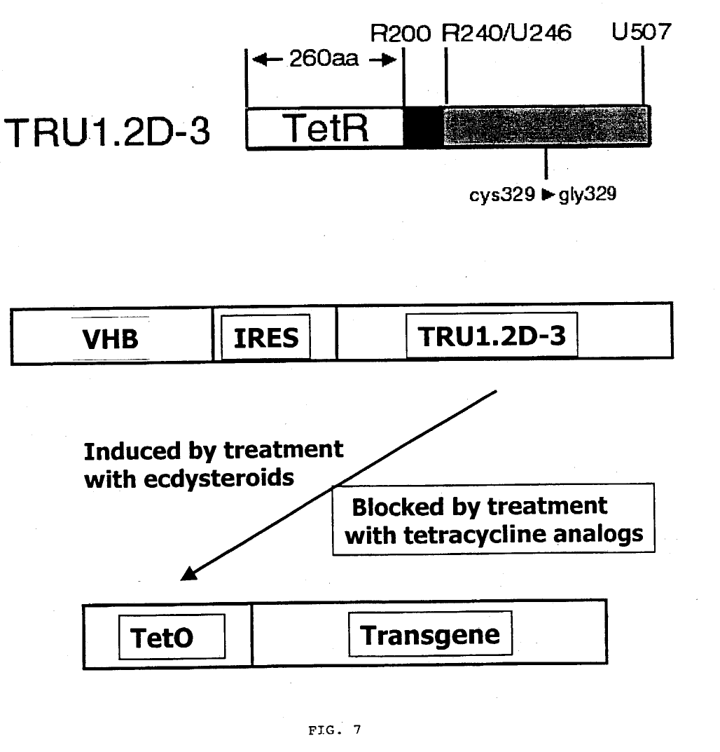

The DNA-binding dimer partner protein fused the 95 amino acid Gal4 DNA binding domain via a BspE I site at the 3′ end to a region of either the RXR or Usp protein with high affinity ligand dependent interaction with BEcR. These variants used amino acids 200-462 of the human RXR alpha protein or amino acids 169-507 of the Drosophila Usp protein (see FIG. 3). These proteins are termed GR (for the RXR variant) or GU (for the Usp variant). GRU fusion chimeras were produced by similar methods and coupled individual regions of both proteins as shown in FIG. 5. Generation of GRU1.2D-3 was performed by PCR-mediated site directed mutagenesis of the GRU1.2 HBD to convert cysteine 329 (numbering in the native Drosophila Usp protein) to a glycine residue (FIG. 6).

Generation of the tetracycline-repressor (TR) DNA binding domain fusion to the RU1.2D-3 domain was performed by PCR of sequences corresponding to the 260 amino acid DNA binding region of TR and fusion in-frame using the BspE I site as in GR and GU above (See FIG. 7).

All constructs were assayed by luciferase expression assay and transient transfection using the Gal4-Tk minimal promoter-luciferase reporter construct GalO-luc. This vector was constructed by insertion of two tandem 17 bp Gal operators into the TK-luc reporter previously described for E4 luc (see Suhr et al., supra).

Transient transfection assays were performed on common cultured cell types using calcium-phosphate precipitation and standard methods as described by Suhr et al., supra. Transfected cells were treated uniformly with 1 micromolar ligand in ethanol vehicle for 48 hrs. after transfection before harvest and assay for activity, as described by Suhr et al., supra.

It will be apparent to those skilled in the art that various changes may be made in the invention without departing from the spirit and scope thereof, and therefore, the invention encompasses embodiments in addition to those specifically disclosed in the specification.

What is claimed is:

1. A system for modulating the expression of a target gene associated with a defined response element in a subject, said system comprising:
a first chimeric protein comprising at least one dimerization domain of a first member of the steroid/thyroid hormone nuclear receptor superfamily and at least one DNA binding domain, and
a second chimeric protein comprising at least one dimerization domain of a second member of the steroid/thyroid hormone nuclear receptor superfamily and at least one transcription modulating domain,
wherein the first and second chimeric proteins associate to form a functional entity under substantially physiological conditions, and
wherein response of said response element to said DNA binding domain modulates expression of said target gene.

2. The system according to claim 1 wherein one or both of the first and second chimeric proteins further comprises at least one dimerization affinity enhancing domain.

3. The system according to claim 1 wherein one or both of the first and second chimeric proteins comprise at least one ligand binding domain.

4. The system according to claim 3 wherein one of the chimeric proteins comprises at least one ligand binding domain.

5. The system according to claim 1 wherein one member contains at least one functional domain of a retinoid X receptor or an Ultraspiracle receptor.

6. The system according to claim 1 wherein the dimerization domain of one member comprises at least one ligand binding domain of an ecdysone receptor.

7. The system according to claim 6 wherein the ecdysone receptor is a Drosophila ecdysone receptor.

8. The system according to claim 1 wherein the dimerization domain of one member comprises at least one ligand binding domain of a Diptera or Lepidoptera receptor.

9. The system according to claim 8 wherein the Lepidoptera receptor is a Bombyx ecdysone receptor.

10. The system according to claim 2 wherein the dimerization affinity enhancing domain is derived from the Bombyx ecdysone receptor.

11. The system according to claim 2 wherein the dimerization affinity enhancing domain is derived from the Drosophila ecdysone receptor.

12. The system according to claim 1 wherein the modulating is activation of expression.

13. The system according to claim 12 wherein the transcription modulating domain is an activation domain selected from the group consisting of VP16τ, GAL4, NF-κB and BP64 activation domains.

14. The system according to claim 13 wherein the activation domain is the VP16τ activation domain.

15. The system according to claim 1 wherein the modulating is repression of expression.

16. The system according to claim 15 wherein the transcription modulating domain is a transcription repressor domain selected from the group consisting of RAFT, CREM, MECP-2, SMRT, NcoR, mSin3A, RAR, TR, and SMRTR.

17. The system according to claim 1 further comprising the response element for said DNA binding domain that modulates expression of said target gene.

18. The system according to claim 17 wherein the target gene is a reporter gene.

19. The system according to claim 1 wherein the DNA binding domain is a Gal4 DNA binding domain.

20. The system according to claim 1 wherein the first dimerization domain comprises at least one ligand binding domain of a retinoid X receptor or an Ultraspiracle receptor.

21. The system according to claim 20 wherein the first dimerization domain comprises at least one ligand binding domain of an ecdysone receptor.

22. The system according to claim 1 wherein the DNA binding domain is a Tet operon.

23. The system according to claim 22 wherein the first dimerization domain comprises at least one ligand binding domain of an ecdysone receptor.

24. The system according to claim 1 wherein the chimeric proteins associate in the presence of ligand for the ligand binding domain and the ligand is a synthetic organic compound, an insect derived steroid, a plant derived steroid, a plant extract or a synthetic steroid.

25. The system according to claim 24 wherein ligand is a steroidal plant extract.

26. The system according to claim 24 wherein the ligand is ecdysone or 20-hydroxy ecdysone.

27. A system for modulating the expression of a target gene associated with a defined response element in a subject, said system comprising:
a first chimeric protein comprising at least one dimerization domain of a first member of the steroid/thyroid hormone nuclear receptor superfamily and at least one DNA binding domain, and
a second chimeric protein comprising at least one dimerization domain of a second member of the steroid/thyroid hormone nuclear receptor superfamily and at least one transcription modulating domain, and
wherein, at least one of the receptors is non-endogenous to said subject and the first and second chimeric proteins associate to form a functional entity under substantially physiological conditions in the presence or absence of a non-endogenous ligand, and
wherein response of said response element to said DNA binding domain modulates expression of said target gene.

28. Isolated nucleic acid encoding the first and second chimeric proteins according to claim 1.

29. The isolated nucleic acid according to claim 28 wherein the first and second chimeric proteins are jointly encoded by the isolated nucleic acid.

30. The isolated nucleic acid according to claim 28 wherein the nucleic acid encodes an internal ribosomal entry site located between the nucleotides encoding the first and second chimeric proteins.

31. The isolated nucleic acid according to claim 30 wherein the first and second chimeric proteins are encoded by separate nucleic acids.

32. A vector system containing isolated nucleic acid encoding the first and second chimeric proteins according to claim 1.

33. A vector system containing isolated nucleic acid encoding the first and second chimeric proteins according to claim 17.

34. A cell containing a vector system according to claim 32.

35. A method for modulating the expression of one or more target genes in a subject containing:
1) a system according to claim 1, and
2) DNA encoding one or more target gene products under the control of a response element to which the DNA binding domain is responsive,
said method comprising subjecting the subject to conditions suitable to induce expression of the system, thereby modulating expression of the one or more target genes.

36. The method according to claim 35 wherein the system is encoded by one or more DNA constructs.

37. The method according to claim 35 wherein the one or more target gene products are non-endogenous.

38. The method according to claim 35 wherein the response element is endogenous.

39. The method according to claim 35 wherein the subject is non-mammalian.

40. The method according to claim 35 wherein the subject is mammalian.

41. A method for modulating the expression of one or more target genes in a subject containing:
1) one or more inducible DNA constructs encoding the system according to claim 1, and
2) DNA encoding one or more target gene products under the control of a response element to which the DNA binding domain is responsive,
said method comprising subjecting said subject to conditions suitable to induce expression of the system, thereby modulating expression of said one or more target genes.

42. A method for modulating the expression of one or more target genes in a cell line containing:
1) a system according to claim 1, and
2) DNA encoding one or more target gene products under the control of a response element to which the DNA binding domain is responsive,
said method comprising administering to the cell line an effective amount of ligand for the system, thereby modulating expression of said one or more target genes.

43. The method according to claim 42 wherein the system is encoded by one or more isolated DNA constructs.

44. The method according to claim 42 wherein the cell line is mammalian.

45. The method according to claim 42 wherein the cell line is non-mammalian.

46. A system for modulating the expression of a target gene target gene associated with a defined response element in a subject, said system comprising:
a first chimeric protein consisting of a DNA binding domain and a dimerization domain of a first member of the steroid/thyroid hormone nuclear receptor superfamily, and
a second chimeric protein consisting of a transcription modulating domain and a dimerization domain of a second member of the steroid/thyroid hormone nuclear receptor superfamily,
wherein at least one of the receptors is non-endogenous and the first and second chimeric proteins associate to form a functional entity under substantially physiological conditions in the presence of a non-endogenous ligand, and
wherein association of said response element with said DNA binding domain modulates expression of said target gene.
